(19) United States
(12) Patent Application Publication
(10) Pub. No.: US 2018/0015139 A1
(43) Pub. Date: Jan. 18, 2018

(54) Low-Dose Stable Formulations of Linaclotide

(71) Applicants: Ironwood Pharmaceuticals, Inc., Cambridge, MA (US); Forest Laboratories Holdings Limited, Hamilton (BM)

(72) Inventors: Yun Mo, Commack, NY (US); Caroline Kurtz, Sudbury, MA (US)

(21) Appl. No.: 15/718,075

(22) Filed: Sep. 28, 2017

Related U.S. Application Data
(63) Continuation of application No. 15/435,701, filed on Feb. 17, 2017, now abandoned.
Application No. 15/435,701 is a continuation of application No. 15/103,634, filed on Jun. 10, 2016, now abandoned.
(60) Provisional application No. 61/914,951, filed on Dec. 11, 2013.
(60) Provisional application No. 61/914,952, filed on Dec. 11, 2013.

(51) Int. Cl. A61K 38/10; A61K 9/16; A61K 9/48

(52) U.S. Cl. CPC A61K 38/10; A61K 9/4866; A61K 9/4858; A61K 9/485; A61K 9/1611; A61K 9/1652; A61K 9/1635; A61K 9/1617; A61K 9/4825

(57) ABSTRACT

The present invention relates to stable pharmaceutical compositions comprising linaclotide or pharmaceutically acceptable salts thereof, as well as to various methods and processes for the preparation and use of the compositions.

### CLAIM OF PRIORITY

This application is a continuation of, and claims priority under 35 U.S.C. §120, to U.S. patent application Ser. No. 15/435,701 filed Feb. 17, 2017, which is a continuation of U.S. patent application Ser. No. 15/103,634 filed Jun. 10, 2016, which is the United States National Phase of PCT/US2014/069851, filed Dec. 11, 2014, which claims priority under 35 U.S.C. §119(e) to U.S. Provisional Application No. 61/914,951 filed on Dec. 11, 2013 and to U.S. Provisional Application No. 61/914,952 filed on Dec. 11, 2013, the entire contents of which are hereby incorporated by reference.

### SEQUENCE LISTING

This application incorporates by reference in its entirety the Sequence Listing entitled “Single_linaclotide_listing_ST25.txt” (570 bytes) which was created Dec. 11, 2014 and filed electronically herewith.

### FIELD OF THE INVENTION

The present invention relates to low-dose stable pharmaceutical compositions of linaclotide and methods for treating gastrointestinal disorders by administering the pharmaceutical compositions.

### BACKGROUND OF THE INVENTION

Various formulation techniques have been used to develop compositions for pharmaceutically active agents. However, the specific components of these compositions vary greatly and depend significantly on the particular pharmaceutically active agent and the desired properties and dosage concentrations. For example, the formulation must be compatible with the pharmaceutically active agent and also provide the necessary stability properties.

U.S. Pat. Nos. 7,304,036 and 7,371,727, herein incorporated by reference, disclose peptides that act as agonists of the guanylate cyclase C (GC-C) receptor for the treatment of gastrointestinal (GI) disorders. One particular peptide disclosed is linaclotide, which consists of the following amino acid sequence: Cys Cys Glu Tyr Cys Cys Asn Pro Ala Cys Thr Gly Cys Tyr. Linaclotide has the chemical structure of:

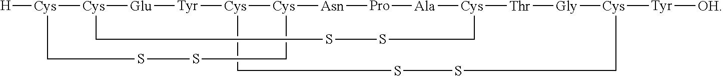

Linaclotide is orally administered and has been approved in the U.S. by the FDA for the treatment of irritable bowel syndrome with constipation (IBS-c) and chronic idiopathic constipation (CIC). In humans, linaclotide has been shown to effect GI physiology including reducing visceral pain, reducing bloating and increasing GI transit which can lead to increased stool frequency and improved stool consistency. Orally administered linaclotide acts locally by binding to and activating GC-C receptors at the luminal surface of the intestine. The GC-C receptor is a key regulator in mammals of intestinal function and is found throughout the luminal surface of the GI tract. The GC-C receptor responds to the endogenous hormones, guanylin and uroguanylin, and to enteric bacterial peptides from the heat stable enterotoxin family (ST peptide). When linaclotide binds to the GC-C receptor, there is an elevation of the second messenger, cyclic GMP (c-GMP), and an increase in chloride and bicarbonate secretion, resulting in an increase in intestinal fluid secretion and reducing pain.

As approved by the FDA, linaclotide is administered in an oral, solid, capsule formulation manufactured by filling drug-layered beads into gelatin capsules. Linaclotide is currently approved for adults in once daily administration at 145 μg for CIC or 290 μg for IBS-c. U.S. Pat. Nos. 8,748,573 and 8,802,628, herein incorporated by reference, disclose the commercial formulation and methods of use thereof.

However, there is a need for low-dose linaclotide formulations, including for example, geriatric and pediatric formulations, which have improved stability and performance. Pediatric and geriatric patients as well as individuals who may be at high risk of adverse reactions (e.g. diarrhea) may benefit from low-dose formulations of linaclotide. Low-dose formulations also may be useful for treating additional disorders for which the current commercial formulations would not be suitable.

The challenge for developing low-dose formulations arises in part because of the intrinsic and chemical instability of linaclotide (for example, induced by moisture-driven degradation reactions such as hydrolysis, deamidation and isomerization). These difficulties may be exacerbated when producing pediatric or geriatric formulations and other low-dose formulations of linaclotide because linaclotide is more dispersed and has greater surface area exposure to aqueous environments during preparation and storage.

The present invention provides improved stable formulations of linaclotide. These formulations are described herein.

### BRIEF DESCRIPTION OF THE FIGURES

FIG. 1 illustrates stability profiles for 36 μg linaclotide compositions through 6 months at 40° C. and 75% relative humidity.

FIG. 2 illustrates stability profiles for 72 μg linaclotide compositions through 6 months at 40° C. and 75% relative humidity.

FIG. 3 shows a normalized overlay of chromatograms showing impurities in a linaclotide formulation sample.

### SUMMARY OF THE INVENTION

In some embodiments of the present invention, a stable pharmaceutical composition is provided which comprises linaclotide, a cation or salt thereof, histidine, and, optionally, a polymer.

In some embodiments, a stable low-dose solid oral dosage form of linaclotide is provided. In some embodiments, a stable pediatric solid oral dosage form of linaclotide is provided.

In some embodiments, the pharmaceutical composition comprises linaclotide, a cation or pharmaceutically acceptable salt thereof and histidine, wherein the composition has a molar ratio of cation:histidine of less than 1:1.

In some embodiments, a stable pharmaceutical composition is provided which comprises linaclotide, a cation or salt thereof, histidine, and, optionally, a polymer.

In some embodiments, a pharmaceutical composition (e.g., granules or beads) is provided which comprises linaclotide, a cation or pharmaceutically acceptable salt thereof, a sterically hindered amine selected from histidine, and a polymer selected from polyvinyl pyrrolidone (PVP), polyvinyl alcohol (PVA) or a mixture thereof.

In some embodiments, a solid oral dosage form (e.g., capsules or tablets) is provided which comprises linaclotide, a cation or pharmaceutically acceptable salt thereof, a sterically hindered amine selected from histidine, and a polymer selected from polyvinyl pyrrolidone (PVP), polyvinyl alcohol (PVA) or a mixture thereof.

In some embodiments, a method of treating a gastrointestinal disorder or other disorder comprising administering to a patient in need thereof, a therapeutically effective amount of the pharmaceutical compositions described above is provided.

### DETAILED DESCRIPTION OF THE INVENTION

Stable formulations of linaclotide (SEQ ID NO:1) are provided herein. In addition, methods of using the formulations to treat gastrointestinal disorders, and processes for making the compositions are provided.

It has been found that the stability of linaclotide within solid oral dosage forms (e.g., capsules and tablets) can be improved by combining linaclotide with specific concentrations or molar ratios of a cation or pharmaceutically acceptable salt thereof, and an amine. In some embodiments, stability may be improved by combining linaclotide with specific concentrations or molar ratios of a polymer, cation or pharmaceutically acceptable salt thereof, and an amine selected from histidine. In some embodiments, stability may be improved by combining linaclotide with specific concentrations of a polymer, a cation selected from Ca²⁺ or a pharmaceutically acceptable salt thereof, and an amine selected from histidine. It has been found, in some embodiments, that combining these components with linaclotide causes an increase or improvement in the stability of linaclotide within the composition, for example as compared to similar compositions not containing the cation and/or sterically hindered amine and/or the same concentrations of these components.

In some embodiments, for example, each solid oral dosage form (e.g., a capsule or tablet) comprises from 0.1 μg to 100 μg of linaclotide. In some embodiments, for example, the solid oral dosage form comprises from 1 μg to 80 μg of linaclotide. In some embodiments, for example, the solid oral dosage form comprises from 2 μg to 75 μg of linaclotide. In some embodiments, for example, the solid oral dosage form comprises from 5 μg to 75 μg of linaclotide. In some embodiments, for example, the solid oral dosage form comprises from 1 μg to 40 μg, 2 μg to 50 μg, or 5 μg to 50 μg of linaclotide. In some embodiments, for example, the solid oral dosage form comprises from 1 μg to 30 μg of linaclotide. In some embodiments, for example, the solid oral dosage form comprises from 1 μg to 20 μg of linaclotide. In some embodiments, for example, the solid oral dosage form comprises from 1 μg to 10 μg of linaclotide.

In some embodiments, the solid oral dosage form comprises 0.1 μg, 0.15 μg, 0.25 μg, 0.5 μg, 0.75 μg, 1 μg, 2.5 μg, 5 μg, 7.5 μg, 9 μg, 10 μg, 15 μg, 18 μg, 20 μg, 30 μg, 36 μg, 40 μg, 50 μg, 60 μg, 72 μg, 80 μg, and 100 μg of linaclotide. In some embodiments, the solid oral dosage form comprises about 72 μg of linaclotide. In some embodiments, the solid oral dosage form comprises about 36 μg of linaclotide. In some embodiments, the solid oral dosage form comprises about 18 μg of linaclotide. In some embodiments, the solid oral dosage form comprises about 10 μg of linaclotide. In some embodiments, the solid oral dosage form comprises about 9 μg of linaclotide.

In some embodiments, the pharmaceutical composition (e.g., bead or granule) comprises 0.001 to 0.5% by weight of linaclotide, for example, 0.001 to 0.1% by weight, 0.03 to 0.09% by weight. In some embodiments, the pharmaceutical composition (e.g., bead or granule) comprises about 0.06% by weight of linaclotide.

In some embodiments, the pharmaceutical composition also comprises histidine, either alone or in combination with another sterically hindered amine. In some embodiments, the other sterically hindered amine is an amino acid. In some embodiments, the other sterically hindered amine is a naturally occurring amino acid. In some embodiments, the naturally occurring amino acid is selected from leucine, isoleucine, methionine or asparagine. In other embodiments, the pharmaceutical composition comprises linaclotide, a cation or pharmaceutically acceptable salt thereof and histidine, wherein the composition has a molar ratio of cation:histidine of less than 2:1. In some embodiments, histidine is replaced in the compositions with asparagine.

In some embodiments, for example, the composition comprises a molar ratio of histidine (or mixture thereof) to linaclotide between 150:1 and 80:1. In some embodiments, for example, the composition comprises a molar ratio of histidine (or mixture thereof) to linaclotide between 120:1 and 80:1. In some embodiments, the composition comprises a molar ratio of histidine (or mixture thereof) to linaclotide between 110:1 and 90:1. In some embodiments, the composition comprises a molar ratio of histidine (or mixture thereof) to linaclotide of about 100:1. In some embodiments, the composition comprises a molar ratio of histidine (or mixture thereof) to linaclotide of at least 40:1. In some embodiments, the composition comprises a molar ratio of histidine (or mixture thereof) to linaclotide of at least 80:1.

In some embodiments, the pharmaceutical composition (e.g., bead or granule) comprises 0.3% to 1.0% by weight of histidine, for example, 0.4% to 0.8% by weight. In some embodiments, the pharmaceutical composition (e.g., bead or granule) comprises about 0.3% by weight of histidine. In some embodiments, the pharmaceutical composition (e.g., bead or granule) comprises about 0.67% by weight of linaclotide.

Suitable cations include, for example, metal or organic cations. In some embodiments, the composition comprises a metal cation selected from calcium, potassium, magnesium, zinc, aluminum, manganese, sodium, or a combination or mixture thereof. In some embodiments, the composition comprises a divalent metal cation. In some embodiments, the composition comprises a divalent metal cation selected from Ca²⁺, Mg²⁺, Zn²⁺, Mn²⁺, or a combination or mixture thereof.

In some embodiments, the composition comprises Ca²⁺.

The cation can be added to the composition in any suitable form, for example any pharmaceutically acceptable salt with any appropriate counterion. Suitable metal salts include, for example, calcium chloride, calcium carbonate, calcium acetate, magnesium chloride, magnesium acetate, zinc acetate, zinc chloride, aluminum chloride or mixtures thereof. In some embodiments, the composition comprises calcium chloride, magnesium chloride, zinc acetate, or a combination or mixture thereof. In some embodiments, the composition comprises calcium chloride.

In some embodiments, the pharmaceutical composition comprises a molar ratio of cation (e.g., Ca²⁺ or a salt thereof) to linaclotide between 70:1 and 30:1. In some embodiments, the composition comprises a molar ratio of cation (e.g., Ca²⁺ or a salt thereof) to linaclotide between 60:1 and 40:1. In some embodiments, the composition comprises a molar ratio of cation (e.g., Ca²⁺ or a salt thereof) to linaclotide is about 50:1. In some embodiments, the composition comprises a molar ratio of cation to linaclotide of less than 80:1. In some embodiments, the composition comprises a molar ratio of cation to linaclotide of less than 60:1.

In some embodiments, the composition (e.g., bead or granule) comprises 0.01 to 10% by weight of Ca²⁺ or a pharmaceutically acceptable salt thereof. In some embodiments, the composition comprises 0.1 to 1.0 wt. % of calcium chloride dihydrate. In some embodiments, the composition comprises 0.25 to 0.40 wt. % of calcium chloride dihydrate. In some embodiments, the composition comprises about 0.32 wt. % of calcium chloride dihydrate.

In some embodiments, the pharmaceutical composition comprises a stabilizing amount of an amino acid selected from histidine and a stabilizing amount of a cation (e.g., a metal cation, for example, a divalent metal cation selected from Mg²⁺, Ca²⁺, Zn²⁺ or a salt thereof or a combination or mixture thereof). In some embodiments, the composition comprises a stabilizing amount of histidine and a stabilizing amount of Ca²⁺ or a salt thereof.

In some embodiments, the composition comprises a cation and amino acid (e.g., histidine or mixture thereof) in a molar ratio of cation:amino acid (e.g., Ca²⁺:histidine) of less than 2:1. In some embodiments, the composition comprises a cation and amino acid (e.g., histidine or mixture thereof) in a molar ratio of cation:amino acid (e.g., Ca²⁺:histidine) of less than 1:1. In some embodiments, the composition comprises a cation and amino acid (e.g., histidine) in a molar ratio of cation:amino acid (e.g., Ca²⁺:histidine) between 1:5 and 1:1. In some embodiments, the composition comprises a cation and amino acid in a molar ratio of cation:amino acid (e.g., Ca²⁺:histidine) between 1:1.5 and 1:2.5. In some embodiments, the composition comprises a cation and amino acid in a molar ratio of cation:amino acid (e.g., Ca²⁺:histidine) between 1:1.8 and 1:2.2. In some embodiments, the composition comprises a cation and amino acid in a molar ratio of cation:amino acid (e.g., Ca²⁺:histidine) between 1:1.9 and 1:2.1. In some embodiments, the composition comprises a cation and amino acid in a molar ratio of cation:amino acid (e.g., Ca²⁺:histidine) of 1:2. In some embodiments, the composition comprises Ca²⁺ or a pharmaceutically acceptable salt thereof and histidine in a molar ratio of Ca²⁺:histidine between 1:1.5 and 1:2.5. In some embodiments, the composition comprises Ca²⁺ or a pharmaceutically acceptable salt thereof and histidine in a molar ratio of Ca²⁺:histidine between 1:1.8 and 1:2.2. In some embodiments, the composition comprises Ca²⁺ or a pharmaceutically acceptable salt thereof and histidine in a molar ratio of Ca²+:histidine between 1:1.9 and 1:2.1. In some embodiments, the composition comprises Ca²⁺ or a pharmaceutically acceptable salt thereof and histidine in a molar ratio of Ca²⁺:histidine of 1:2.

In some embodiments, the composition comprises a cation, amino acid and linaclotide in a molar ratio of cation:amino acid:linaclotide (e.g., Ca²⁺:histidine:linaclotide) of between 30:80:1 and 80:150:1. In some embodiments, the composition comprises a cation, amino acid and linaclotide in a molar ratio of cation:amino acid:linaclotide (e.g., Ca²⁺:histidine:linaclotide) of between 30:80:1 and 70:120:1. In some embodiments, the composition comprises a cation, amino acid and linaclotide in a molar ratio of cation:amino acid:linaclotide (e.g., Ca²⁺:histidine:linaclotide) of between 40:90:1 and 60:110:1.

In some embodiments, the composition comprises Ca²⁺ or a pharmaceutically acceptable salt thereof, histidine and linaclotide in a molar ratio of Ca²⁺:histidine:linaclotide of between 40:90:1 and 60:110:1. In some embodiments, the composition comprises Ca²⁺ or a pharmaceutically acceptable salt thereof, histidine and linaclotide in a molar ratio of Ca²⁺:histidine:linaclotide of between 45:95:1 and 55:105:1. In some embodiments, the composition comprises Ca²⁺ or a pharmaceutically acceptable salt thereof, histidine and linaclotide in a molar ratio of Ca²⁺:histidine:linaclotide of 50:100:1.

Suitable polymers include, for example, polyvinyl pyrrolidone (PVP), polyvinyl alcohol (PVA), hydroxylpropyl methyl cellulose (HPMC), hydroxylpropyl cellulose (HPC), methyl cellulose, methacrylate polymers, cyclodextrin, dextrin, dextran, polyacrylic acid, chitosan, guar gum, xanthan gum, polyethylene oxide (e.g., polyethylene polypropylene oxide), poly (sodium vinylsulfonate), polyethylene glycol, poly(arginine), poly carbophil, polyvinyl pyrrolidone-co-vinyl acetate, a poloxamer (e.g., Pluronic® products available from BASF), alginate, trehalose, sucrose, inulin, or a combination or mixture thereof. In some embodiments, the composition comprises a polymer selected from PVP, PVA, methacrylate polymers, cyclodextrin, dextran, polyacrylic acid, chitosan, guar gum, xanthan gum, polyethylene oxide, polyethylene glycol, poly(arginine), poly carbophil, polyvinyl pyrrolidone-co-vinyl acetate, a poloxamer, or a combination or mixture thereof. In some embodiments, the composition comprises PVP, PVA, polyethylene oxide, or a mixture thereof. In some embodiments, the composition comprises PVP, PVA, or a mixture thereof. In some embodiments, the composition comprises PVP. In some embodiments, the composition comprises PVA.

In some embodiments, the composition (e.g., bead or granule) comprises 0.1 to 10% by weight of a polymer (for example, PVA or PVP). In some embodiments, the composition comprises 1 to 5 wt. % of a polymer component, wherein the polymer component is PVA or PVP. In some embodiments, the composition comprises 1 to 3 wt. % of a polymer component, wherein the polymer component is PVA. In some embodiments, the composition comprises about 1.5 wt. % of a polymer (e.g., PVA or PVP). In some embodiments, the composition comprises about 1.5 wt. % of PVA.

In some embodiments, the pharmaceutical composition comprises (i) a polymer (e.g., PVP or PVA), (ii) a stabilizing amount of histidine, and (iii) a stabilizing amount of a cation (e.g., a divalent metal cation for example Ca²⁺ or a pharmaceutically-acceptable salt thereof). In some embodiments, the composition comprises a stabilizing amount of PVA and stabilizing amounts of histidine and Ca²⁺.

In some embodiments, the composition comprises 1 to 5 wt % of PVA, Ca²⁺ or a pharmaceutically acceptable salt thereof, histidine and linaclotide in a molar ratio of Ca²⁺:histidine:linaclotide of between 40:90:1 and 60:110:1. In some embodiments, the composition comprises 1 to 3 wt % of PVA, Ca²⁺ or a pharmaceutically acceptable salt thereof, histidine and linaclotide in a molar ratio of Ca²⁺:histidine:linaclotide of between 45:95:1 and 55:105:1. In some embodiments, the composition comprises 1.5 wt % of PVA, Ca²⁺ or a pharmaceutically acceptable salt thereof, histidine and linaclotide in a molar ratio of Ca²⁺:histidine:linaclotide of 50:100:1.

The pharmaceutical composition may also comprise any one or more processing aids. Suitable processing aids include, but are not limited to, talc, starch, calcium carbonate, calcium sulfate, hydroxylpropylmethyl cellulose, fructose, methyl cellulose, dextrates, dextrose, dextran, lactitol, maltose, sucrose, sorbitol, isomalt, pregelatinized starch, dicalcium phosphate, microcrystalline cellulose, mannitol, gelatin, trehalose, erythitol, maltitol, lactose, glucose, or a combination thereof, or a mixture thereof. In some embodiments, the processing aid is talc. In some embodiments, the processing aid (e.g., talc) is mixed with a composition comprising linaclotide, histidine, Ca²⁺ or pharmaceutically acceptable salt thereof, and optional polymer. In some embodiments, the composition comprises 0.1 to 5 wt % talc. In some embodiments, the composition comprises 0.1 to 1 wt % talc. In some embodiments, the composition comprises about 0.5 wt % talc.The pharmaceutical composition may also comprise any one or more filling agents. Suitable filling agents include, but are not limited to, talc, starch, calcium carbonate, calcium sulfate, hydroxylpropylmethyl cellulose, fructose, methyl cellulose, dextrates, dextrose, dextran, lactitol, maltose, sucrose, sorbitol, isomalt, pregelatinized starch, dicalcium phosphate, microcrystalline cellulose, mannitol, gelatin, trehalose, erythitol, maltitol, lactose, glucose, or a combination thereof, or a mixture thereof. In some embodiments, the filling agent is isomalt. In some embodiments, the filling agent is gelatin. In some embodiments, the filling agent is mannitol. In some embodiments, the filling agent is pregelatinized starch. In some embodiments, the filling agent is microcrystalline cellulose. In some embodiments, a composition comprising the linaclotide, histidine, Ca²⁺ or pharmaceutically acceptable salt thereof, and optional polymer and optional processing aid is mixed with the filling agent. In some embodiments, a composition comprising the linaclotide, histidine, Ca²⁺ or pharmaceutically acceptable salt thereof, and optional polymer is sprayed or layered on the filling agent.

The pharmaceutical composition can comprise any suitable concentration of filling agent. In some embodiments, for example, the composition comprises one or more filling agents in a concentration of 0.1-99% by weight, relative to the total weight of the composition. In some embodiments, for example, the composition comprises one or more filling agents in a concentration of 1-95 wt. % of filling agent(s), relative to the total weight of the composition. In some embodiments, for example, the composition comprises one or more filling agents in a concentration of 10-90 wt. % of filling agent(s), relative to the total weight of the composition. In some embodiments, for example, the composition comprises one or more filling agents in a concentration of 20-90 wt. % of filling agent(s), relative to the total weight of the composition. In some embodiments, the composition comprises one or more filling agents in a concentration of at least 20 wt. %, for example, at least 40 wt. %, at least 60 wt. %, at least 70 wt. %, at least 80 wt. %, at least 90 wt. %, or at least 96% relative to the total weight of the composition.

In some embodiments, the pharmaceutical composition (e.g., orally disintegrating composition) can comprise one or more plasticizers. Suitable plasticizers include, but are not limited to, polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the concentration of the plasticizer in the formulation may be about 0 to about 30 wt %, for example, about 1 to about 20 wt %, about 0.1 to about 10 wt %, about 1 to about 5 wt %, or even 0.1 to about 4 wt %.

One skilled in the art, with the benefit of this disclosure, will understand that other components may be included to enhance one or more properties of the pharmaceutical compositions. In some embodiments, for example, the pharmaceutical composition may include one or more disintegrants, lubricants, anti-caking additives, anti-microbial agents, antifoaming agents, emulsifiers, surfactants, buffering agents, and/or coloring agents.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof. In some embodiments, the disintegrant is crospovidone. In some embodiments, the disintegrant is croscarmellose sodium.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Evonik Degussa Co., Plano, Tex. USA), a pyrogenic silicon dioxide (CAB-O-SIL, Cabot Co., Boston, Mass. USA), and mixtures thereof.

Suitable anti-caking additives include, for example, calcium silicate, magnesium silicate, silicon dioxide, colloidal silicon dioxide, talc, and mixtures thereof. In some embodiments, the composition comprises about 0.01 wt. % to about 5 wt. % of an anti-caking additive (e.g., talc). In some embodiments, the composition comprises about 0.05 wt. % to about 2 wt. % of an anti-caking additive (e.g., talc). In some embodiments, the composition comprises about 0.1 wt. % to about 1 wt. % of an anti-caking additive (e.g., talc). In some embodiments, the composition comprises about 0.25 wt. % to about 0.75 wt. % (e.g., about 0.5 wt. %) of an anti-caking additive (e.g., talc).

Suitable anti-microbial additives that may be used, e.g., as a preservative for the linaclotide compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

In some embodiments, the pharmaceutical composition (e.g., orally-disintegrating composition) may comprise a taste-masking agent. Generally, any natural or synthetic flavoring agent or sweetening agent known in the art may be used in the pharmaceutical compositions of the present invention. For example, suitable taste-masking agents include, but are not limited to, essential oils, water soluble extracts, sugar, monosaccharides, oligosaccharides, aldose, ketose, dextrose, maltose, lactose, glucose, fructose, sucrose, mannitol xylitol, D-sorbitol, erythritol, pentitol, hexitol, malitol, acesulfame potassium, talin, glycyrrhizin, sucralose, aspartame, saccharin, sodium saccharin, sodium cyclamate, eugenyl formate aldehyde flavorings and combinations thereof.

Exemplary aldehyde flavorings that may be used include, but are not limited to acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, L e., melonal (melon); 2-6-dimethyloctanal (green fruit); and 2-dodecenal (citrus, mandarin). In some embodiments, the taste-masking agents may include combination of acesulfame potassium and flavors. One skilled in the art with the benefit of the present disclosure will appreciate that other and further ingredients may be included in the pharmaceutical composition of the present invention, for example, a matrix-forming polymer permeation enhancer, substance for imparting mucoadhesive properties, or other auxiliary substances.

The composition may also comprise any suitable pharmaceutically acceptable carrier or medium. Suitable pharmaceutically acceptable carriers include, for example, any solvents, dispersants, pH-buffering agents, coatings, absorption-promoting agents, controlled-release agents, and one or more inert excipients (e.g., filling agents, starches, polyols, granulating agents, microcrystalline cellulose, diluents, lubricants, binders, disintegrating agents), or the like. In addition, the compositions can contain any desired additional components, additives, and/or species, for example, surface active additives, dispersing additives, humectants, suspending agents, solubilizers, buffering agents, disintegrants, preservatives, colorants, flavorants, and the like. In some embodiments, the composition comprises one or more ion species that interact with linaclotide.

The composition can also comprise any suitable pH buffering agent. In some embodiments, the pH buffering agent is present in the composition in an amount sufficient to achieve the isoelectric point of linaclotide. In the regard, the composition can have any desired pH. In some embodiments, the composition has a pH of 2 to 5 (for example, a pH of 2 to 4.5, a pH of 2 4o 4, a pH of 2.5 to 4, a pH of 2.5 to 3.5, a pH of 2.5 to 3, or even a pH of 3).

In some embodiments, the composition comprises linaclotide and a hydrolysis product, e.g., a hydrolysis product comprising or having a structure of:

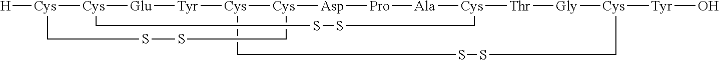

The composition can contain any desired concentration of the hydrolysis product. In some embodiments, the composition comprises less than 10 wt. % of the hydrolysis product. In some embodiments, the composition comprises less than 7 wt. % of the hydrolysis product. In some embodiments, the composition comprises less than 6 wt. % of the hydrolysis product. In some embodiments, the composition comprises less than 5 wt. % of the hydrolysis product. In some embodiments, the composition comprises less than 4 wt. % of the hydrolysis product. In some embodiments, the composition comprises less than 3 wt. % of the hydrolysis product. In some embodiments, the composition comprises less than 2 wt. % of the hydrolysis product. In some embodiments, the composition comprises less than 1 wt. % of the hydrolysis product. In some embodiments, the composition comprises between 0.01 and 10 wt. % of the hydrolysis product. In some embodiments, the composition comprises between 0.1 and 7 wt. % of the hydrolysis product. In some embodiments, the composition comprises between 0.1 and 5 wt. % of the hydrolysis product. In some embodiments, the composition comprises between 0.5 and 5 wt. % of the hydrolysis product. In some embodiments, the composition comprises between 1 and 5 wt. % of the hydrolysis product. In some embodiments, the composition comprises between 0.1 and 4 wt. % of the hydrolysis product. In some embodiments, the composition comprises between 0.5 and 4 wt. % of the hydrolysis product. In some embodiments, the composition comprises between 1 and 4 wt. % of the hydrolysis product. In some embodiments, the composition comprises between 0.1 and 3 wt. % of the hydrolysis product. In some embodiments, the composition comprises between 0.5 and 3 wt. % of the hydrolysis product. In some embodiments, the composition comprises between 1 and 3 wt. % of the hydrolysis product. In some embodiments, the composition comprises between 0.1 and 2.5 wt. % of the hydrolysis product. In some embodiments, the composition comprises between 0.5 and 2.5 wt. % of the hydrolysis product. In some embodiments, the composition comprises between 1 and 2.5 wt. % of the hydrolysis product. In some embodiments, the composition comprises between 0.1 and 2 wt. % of the hydrolysis product. In some embodiments, the composition comprises between 0.5 and 2 wt. % of the hydrolysis product. In some embodiments, the composition comprises between 1 and 2 wt. % of the hydrolysis product. In some embodiments, the composition comprises between 0.1 and 1.5 wt. % of the hydrolysis product. In some embodiments, the composition comprises between 0.5 and 1.5 wt. % of the hydrolysis product. In some embodiments, the composition comprises between 0.1 and 1 wt. % of the hydrolysis product. In some embodiments, the composition comprises between 0.5 and 1 wt. % of the hydrolysis product.

In some embodiments, the composition comprises linaclotide and a peptide modified with the addition of methylene at the α-amine group of the N-terminal Cys₁ that is cross-linked to the amine group of Cys₂ to form an imidazolidinone 5 membered ring at the N-terminus of the peptide (“Cys₁-IMD product”) comprising or having a structure of:

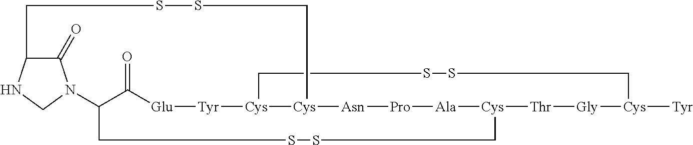

The composition can contain any desired concentration of the Cys₁-IMD product. In some embodiments, the composition comprises less than 10 wt. % of the Cys₁-IMD product. In some embodiments, the composition comprises less than 7 wt. % of the Cys₁-IMD product. In some embodiments, the composition comprises less than 6 wt. % of the Cys₁-IMD product. In some embodiments, the composition comprises less than 5 wt. % of the Cys₁-IMD product. In some embodiments, the composition comprises less than 4 wt. % of the Cys₁-IMD product. In some embodiments, the composition comprises less than 3 wt. % of the Cys₁-IMD product. In some embodiments, the composition comprises less than 2 wt. % of the Cys₁-IMD product. In some embodiments, the composition comprises less than 1 wt. % of the Cys₁-IMD product. In some embodiments, the composition comprises between 0.01 and 10 wt. % of the Cys₁-IMD product. In some embodiments, the composition comprises between 0.1 and 7 wt. % of the Cys₁-IMD product. In some embodiments, the composition comprises between 0.1 and 5 wt. % of the Cys₁-IMD product. In some embodiments, the composition comprises between 0.5 and 5 wt. % of the Cys₁-IMD product. In some embodiments, the composition comprises between 1 and 5 wt. % of the Cys₁-IMD product. In some embodiments, the composition comprises between 0.1 and 4 wt. % of the Cys₁-IMD product. In some embodiments, the composition comprises between 0.5 and 4 wt. % of the Cys₁-IMD product. In some embodiments, the composition comprises between 1 and 4 wt. % of the Cys₁-IMD product. In some embodiments, the composition comprises between 0.1 and 3 wt. % of the Cys₁-IMD product. In some embodiments, the composition comprises between 0.5 and 3 wt. % of the Cys₁-IMD product. In some embodiments, the composition comprises between 1 and 3 wt. % of the Cys₁-IMD product. In some embodiments, the composition comprises between 0.1 and 2.5 wt. % of the Cys₁-IMD product. In some embodiments, the composition comprises between 0.5 and 2.5 wt. % of the Cys₁-IMD product. In some embodiments, the composition comprises between 1 and 2.5 wt. % of the Cys₁-IMD product. In some embodiments, the composition comprises between 0.1 and 2 wt. % of the Cys₁-IMD product. In some embodiments, the composition comprises between 0.5 and 2 wt. % of the Cys₁-IMD product. In some embodiments, the composition comprises between 1 and 2 wt. % of the Cys₁-IMD product. In some embodiments, the composition comprises between 0.1 and 1.5 wt. % of the Cys₁-IMD product. In some embodiments, the composition comprises between 0.5 and 1.5 wt. % of the Cys₁-IMD product. In some embodiments, the composition comprises between 0.1 and 1 wt. % of the Cys₁-IMD product. In some embodiments, the composition comprises between 0.5 and 1 wt. % of the Cys₁-IMD product.

In some embodiments, the composition comprises linaclotide and an oxidation product, e.g., an oxidation product comprising or having a structure of:

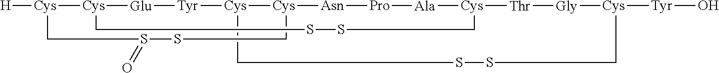

Alternatively, or in addition, the composition comprises linaclotide and an oxidation product having the depicted structure but wherein oxidation occurs at any one or more of the six depicted cysteinyl sulfurs. The composition can contain any desired concentration of the oxidation product. In some embodiments, the composition comprises less than 10 wt. % of the oxidation product. In some embodiments, the composition comprises less than 7 wt. % of the oxidation product. In some embodiments, the composition comprises less than 6 wt. % of the oxidation product. In some embodiments, the composition comprises less than 5 wt. % of the oxidation product. In some embodiments, the composition comprises less than 4 wt. % of the oxidation product. In some embodiments, the composition comprises less than 3 wt. % of the oxidation product. In some embodiments, the composition comprises less than 2 wt. % of the oxidation product. In some embodiments, the composition comprises less than 1 wt. % of the oxidation product. In some embodiments, the composition comprises between 0.01 and 10 wt. % of the oxidation product. In some embodiments, the composition comprises between 0.1 and 7 wt. % of the oxidation product. In some embodiments, the composition comprises between 0.1 and 5 wt. % of the oxidation product. In some embodiments, the composition comprises between 0.5 and 5 wt. % of the oxidation product. In some embodiments, the composition comprises between 1 and 5 wt. % of the oxidation product. In some embodiments, the composition comprises between 0.1 and 4 wt. % of the oxidation product. In some embodiments, the composition comprises between 0.5 and 4 wt. % of the oxidation product. In some embodiments, the composition comprises between 1 and 4 wt. % of the oxidation product. In some embodiments, the composition comprises between 0.1 and 3 wt. % of the oxidation product. In some embodiments, the composition comprises between 0.5 and 3 wt. % of the oxidation product. In some embodiments, the composition comprises between 1 and 3 wt. % of the oxidation product. In some embodiments, the composition comprises between 0.1 and 2.5 wt. % of the oxidation product. In some embodiments, the composition comprises between 0.5 and 2.5 wt. % of the oxidation product. In some embodiments, the composition comprises between 1 and 2.5 wt. % of the oxidation product. In some embodiments, the composition comprises between 0.1 and 2 wt. % of the oxidation product. In some embodiments, the composition comprises between 0.5 and 2 wt. % of the oxidation product. In some embodiments, the composition comprises between 1 and 2 wt. % of the oxidation product. In some embodiments, the composition comprises between 0.1 and 1.5 wt. % of the oxidation product. In some embodiments, the composition comprises between 0.5 and 1.5 wt. % of the oxidation product. In some embodiments, the composition comprises between 0.1 and 1 wt. % of the oxidation product. In some embodiments, the composition comprises between 0.5 and 1 wt. % of the oxidation product.

In some embodiments, the composition comprises linaclotide and an acetylation product, e.g., an acetylation product comprising or having a structure of:

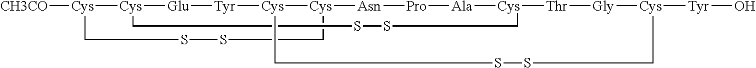

The composition can contain any desired concentration of the acetylation product. In some embodiments, the composition comprises less than 10 wt. % of the acetylation product. In some embodiments, the composition comprises less than 7 wt. % of the acetylation product. In some embodiments, the composition comprises less than 6 wt. % of the acetylation product. In some embodiments, the composition comprises less than 5 wt. % of the acetylation product. In some embodiments, the composition comprises less than 4 wt. % of the acetylation product. In some embodiments, the composition comprises less than 3 wt. % of the acetylation product. In some embodiments, the composition comprises less than 2 wt. % of the acetylation product. In some embodiments, the composition comprises less than 1 wt. % of the acetylation product. In some embodiments, the composition comprises between 0.01 and 10 wt. % of the acetylation product. In some embodiments, the composition comprises between 0.1 and 7 wt. % of the acetylation product. In some embodiments, the composition comprises between 0.1 and 5 wt. % of the acetylation product. In some embodiments, the composition comprises between 0.5 and 5 wt. % of the acetylation product. In some embodiments, the composition comprises between 1 and 5 wt. % of the acetylation product. In some embodiments, the composition comprises between 0.1 and 4 wt. % of the acetylation product. In some embodiments, the composition comprises between 0.5 and 4 wt. % of the acetylation product. In some embodiments, the composition comprises between 1 and 4 wt. % of the acetylation product. In some embodiments, the composition comprises between 0.1 and 3 wt. % of the acetylation product. In some embodiments, the composition comprises between 0.5 and 3 wt. % of the acetylation product. In some embodiments, the composition comprises between 1 and 3 wt. % of the acetylation product. In some embodiments, the composition comprises between 0.1 and 2.5 wt. % of the acetylation product. In some embodiments, the composition comprises between 0.5 and 2.5 wt. % of the acetylation product. In some embodiments, the composition comprises between 1 and 2.5 wt. % of the acetylation product. In some embodiments, the composition comprises between 0.1 and 2 wt. % of the acetylation product. In some embodiments, the composition comprises between 0.5 and 2 wt. % of the acetylation product. In some embodiments, the composition comprises between 1 and 2 wt. % of the acetylation product. In some embodiments, the composition comprises between 0.1 and 1.5 wt. % of the acetylation product. In some embodiments, the composition comprises between 0.5 and 1.5 wt. % of the acetylation product. In some embodiments, the composition comprises between 0.1 and 1 wt. % of the acetylation product. In some embodiments, the composition comprises between 0.5 and 1 wt. % of the acetylation product.

In some embodiments, the composition comprises linaclotide and any desired concentration of a ketone product having the structure:

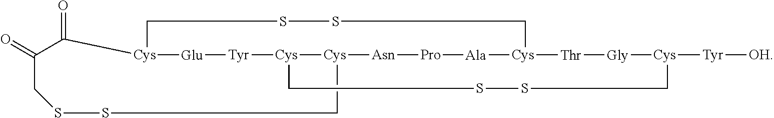

One skilled in the art will recognize that this ketone product could be in equilibrium with its geminal diol monohydrate form having the structure:

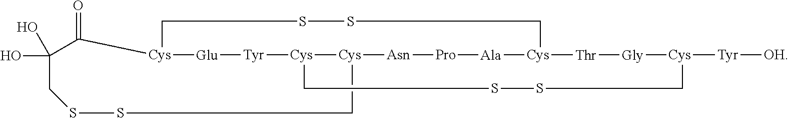

As used herein, the term Cys¹-ketone will be used to refer to both forms.

In some embodiments, the composition comprises less than 10 wt. % of Cys¹-ketone. In some embodiments, the composition comprises less than 7 wt. % of Cys¹-ketone. In some embodiments, the composition comprises less than 6 wt. % of Cys¹-ketone. In some embodiments, the composition comprises less than 5 wt. % of Cys¹-ketone. In some embodiments, the composition comprises less than 4 wt. % of Cys¹-ketone. In some embodiments, the composition comprises less than 3 wt. % of Cys¹-ketone. In some embodiments, the composition comprises less than 2 wt. % of Cys¹-ketone. In some embodiments, the composition comprises less than 1 wt. % of Cys¹-ketone. In some embodiments, the composition comprises between 0.01 and 10 wt. % of Cys¹-ketone. In some embodiments, the composition comprises between 0.1 and 7 wt. % of Cys¹-ketone. In some embodiments, the composition comprises between 0.1 and 5 wt. % of Cys¹-ketone. In some embodiments, the composition comprises between 0.5 and 5 wt. % of Cys¹-ketone. In some embodiments, the composition comprises between 1 and 5 wt. % of Cys¹-ketone. In some embodiments, the composition comprises between 0.1 and 4 wt. % of Cys¹-ketone. In some embodiments, the composition comprises between 0.5 and 4 wt. % of Cys¹-ketone. In some embodiments, the composition comprises between 1 and 4 wt. % of Cys¹-ketone. In some embodiments, the composition comprises between 0.1 and 3 wt. % of Cys¹-ketone. In some embodiments, the composition comprises between 0.5 and 3 wt. % of Cys¹-ketone. In some embodiments, the composition comprises between 1 and 3 wt. % of Cys¹-ketone. In some embodiments, the composition comprises between 0.1 and 2.5 wt. % of Cys¹-ketone. In some embodiments, the composition comprises between 0.5 and 2.5 wt. % of Cys¹-ketone. In some embodiments, the composition comprises between 1 and 2.5 wt. % of Cys¹-ketone. In some embodiments, the composition comprises between 0.1 and 2 wt. % of Cys¹-ketone. In some embodiments, the composition comprises between 0.5 and 2 wt. % of Cys¹-ketone. In some embodiments, the composition comprises between 1 and 2 wt. % of Cys¹-ketone. In some embodiments, the composition comprises between 0.1 and 1.5 wt. % of Cys¹-ketone. In some embodiments, the composition comprises between 0.5 and 1.5 wt. % of Cys¹-ketone. In some embodiments, the composition comprises between 0.1 and 1 wt. % of Cys¹-ketone. In some embodiments, the composition comprises between 0.5 and 1 wt. % of Cys¹-ketone.

In some embodiments, the composition comprises linaclotide and any desired concentration of linaclotide trisulfide, wherein the linaclotide molecule comprises an additional sulfur atom attached to any one of the six cysteinyl sulfurs.

In some embodiments, the composition comprises less than 10 wt. % of linaclotide trisulfide. In some embodiments, the composition comprises less than 7 wt. % of linaclotide trisulfide. In some embodiments, the composition comprises less than 6 wt. % of linaclotide trisulfide. In some embodiments, the composition comprises less than 5 wt. % of linaclotide trisulfide. In some embodiments, the composition comprises less than 4 wt. % of linaclotide trisulfide. In some embodiments, the composition comprises less than 3 wt. % of linaclotide trisulfide. In some embodiments, the composition comprises less than 2 wt. % of linaclotide trisulfide. In some embodiments, the composition comprises less than 1 wt. % of linaclotide trisulfide. In some embodiments, the composition comprises between 0.01 and 10 wt. % of linaclotide trisulfide. In some embodiments, the composition comprises between 0.1 and 7 wt. % of linaclotide trisulfide. In some embodiments, the composition comprises between 0.1 and 5 wt. % of linaclotide trisulfide. In some embodiments, the composition comprises between 0.5 and 5 wt. % of linaclotide trisulfide. In some embodiments, the composition comprises between 1 and 5 wt. % of linaclotide trisulfide. In some embodiments, the composition comprises between 0.1 and 4 wt. % of linaclotide trisulfide. In some embodiments, the composition comprises between 0.5 and 4 wt. % of linaclotide trisulfide. In some embodiments, the composition comprises between 1 and 4 wt. % of linaclotide trisulfide. In some embodiments, the composition comprises between 0.1 and 3 wt. % of linaclotide trisulfide. In some embodiments, the composition comprises between 0.5 and 3 wt. % of linaclotide trisulfide. In some embodiments, the composition comprises between 1 and 3 wt. % of linaclotide trisulfide. In some embodiments, the composition comprises between 0.1 and 2.5 wt. % of linaclotide trisulfide. In some embodiments, the composition comprises between 0.5 and 2.5 wt. % of linaclotide trisulfide. In some embodiments, the composition comprises between 1 and 2.5 wt. % of linaclotide trisulfide. In some embodiments, the composition comprises between 0.1 and 2 wt. % of linaclotide trisulfide. In some embodiments, the composition comprises between 0.5 and 2 wt. % of linaclotide trisulfide. In some embodiments, the composition comprises between 1 and 2 wt. % of linaclotide trisulfide. In some embodiments, the composition comprises between 0.1 and 1.5 wt. % of linaclotide trisulfide. In some embodiments, the composition comprises between 0.5 and 1.5 wt. % of linaclotide trisulfide. In some embodiments, the composition comprises between 0.1 and 1 wt. % of linaclotide trisulfide. In some embodiments, the composition comprises between 0.5 and 1 wt. % of linaclotide trisulfide.

In some embodiments, the composition comprises linaclotide and any desired concentration of a peptide (Des-Tyr14) or a pharmaceutically acceptable salt thereof, wherein the peptide comprises the structure:

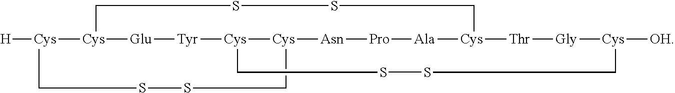

In some embodiments, the composition comprises less than 10 wt. % of Des-Tyr14. In some embodiments, the composition comprises less than 7 wt. % of Des-Tyr14. In some embodiments, the composition comprises less than 6 wt. % of Des-Tyr14. In some embodiments, the composition comprises less than 5 wt. % of Des-Tyr14. In some embodiments, the composition comprises less than 4 wt. % of Des-Tyr14. In some embodiments, the composition comprises less than 3 wt. % of Des-Tyr14. In some embodiments, the composition comprises less than 2 wt. % of Des-Tyr14. In some embodiments, the composition comprises less than 1 wt. % of Des-Tyr14. In some embodiments, the composition comprises between 0.01 and 10 wt. % of Des-Tyr14. In some embodiments, the composition comprises between 0.1 and 7 wt. % of Des-Tyr14. In some embodiments, the composition comprises between 0.1 and 5 wt. % of Des-Tyr14. In some embodiments, the composition comprises between 0.5 and 5 wt. % of Des-Tyr14. In some embodiments, the composition comprises between 1 and 5 wt. % of Des-Tyr14. In some embodiments, the composition comprises between 0.1 and 4 wt. % of Des-Tyr14. In some embodiments, the composition comprises between 0.5 and 4 wt. % of Des-Tyr14. In some embodiments, the composition comprises between 1 and 4 wt. % of Des-Tyr14. In some embodiments, the composition comprises between 0.1 and 3 wt. % of Des-Tyr14. In some embodiments, the composition comprises between 0.5 and 3 wt. % of Des-Tyr14. In some embodiments, the composition comprises between 1 and 3 wt. % of Des-Tyr14. In some embodiments, the composition comprises between 0.1 and 2.5 wt. % of Des-Tyr14. In some embodiments, the composition comprises between 0.5 and 2.5 wt. % of Des-Tyr14. In some embodiments, the composition comprises between 1 and 2.5 wt. % of Des-Tyr14. In some embodiments, the composition comprises between 0.1 and 2 wt. % of Des-Tyr14. In some embodiments, the composition comprises between 0.5 and 2 wt. % of Des-Tyr14. In some embodiments, the composition comprises between 1 and 2 wt. % of Des-Tyr14. In some embodiments, the composition comprises between 0.1 and 1.5 wt. % of Des-Tyr14. In some embodiments, the composition comprises between 0.5 and 1.5 wt. % of Des-Tyr14. In some embodiments, the composition comprises between 0.1 and 1 wt. % of Des-Tyr14. In some embodiments, the composition comprises between 0.5 and 1 wt. % of Des-Tyr14.

In some embodiments, the composition comprises linaclotide and any desired concentration of multimers. In some embodiments, the composition comprises less than 10 wt. % of multimer(s). In some embodiments, the composition comprises less than 7 wt. % of multimer(s). In some embodiments, the composition comprises less than 6 wt. % of multimer(s). In some embodiments, the composition comprises less than 5 wt. % of multimer(s). In some embodiments, the composition comprises less than 4 wt. % of multimer(s). In some embodiments, the composition comprises less than 3 wt. % of multimer(s). In some embodiments, the composition comprises less than 2 wt. % of multimer(s). In some embodiments, the composition comprises less than 1 wt. % of multimer(s). In some embodiments, the composition comprises between 0.01 and 10 wt. % of multimer(s). In some embodiments, the composition comprises between 0.1 and 7 wt. % of multimer(s). In some embodiments, the composition comprises between 0.1 and 5 wt. % of multimer(s). In some embodiments, the composition comprises between 0.5 and 5 wt. % of multimer(s). In some embodiments, the composition comprises between 1 and 5 wt. % of multimer(s). In some embodiments, the composition comprises between 0.1 and 4 wt. % of multimer(s). In some embodiments, the composition comprises between 0.5 and 4 wt. % of multimer(s). In some embodiments, the composition comprises between 1 and 4 wt. % of multimer(s). In some embodiments, the composition comprises between 0.1 and 3 wt. % of multimer(s). In some embodiments, the composition comprises between 0.5 and 3 wt. % of multimer(s). In some embodiments, the composition comprises between 1 and 3 wt. % of multimer(s). In some embodiments, the composition comprises between 0.1 and 2.5 wt. % of multimer(s). In some embodiments, the composition comprises between 0.5 and 2.5 wt. % of multimer(s). In some embodiments, the composition comprises between 1 and 2.5 wt. % of multimer(s). In some embodiments, the composition comprises between 0.1 and 2 wt. % of multimer(s). In some embodiments, the composition comprises between 0.5 and 2 wt. % of multimer(s). In some embodiments, the composition comprises between 1 and 2 wt. % of multimer(s). In some embodiments, the composition comprises between 0.1 and 1.5 wt. % of multimer(s). In some embodiments, the composition comprises between 0.5 and 1.5 wt. % of multimer(s). In some embodiments, the composition comprises between 0.1 and 1 wt. % of multimer(s). In some embodiments, the composition comprises between 0.5 and 1 wt. % of multimer(s).

In some embodiments, the composition comprises linaclotide and one or more products selected from the hydrolysis product, the Cys¹-IMD product, the oxidation product, the Cys¹-ketone product, the acetylation product, the trisulfide product, the Des-Tyr¹⁴ product and the multimer(s).

In some embodiments, the composition comprises a total degradant concentration of less than about 10 wt. %. In some embodiments, the composition comprises a total degradant concentration of less than about 8 wt. %. In some embodiments, the composition comprises a total degradant concentration of less than about 7 wt. %. In some embodiments, the composition comprises a total degradant concentration of less than about 6.5 wt. %. In some embodiments, the composition comprises a total degradant concentration of less than about 6 wt. %. In some embodiments, the composition comprises a total degradant concentration of less than about 5.5 wt. %. In some embodiments, the composition comprises a total degradant concentration of less than about 5 wt. %. In some embodiments, the composition comprises a total degradant concentration of less than about 4 wt. %. In some embodiments, the composition comprises a total degradant concentration of less than about 3 wt. %. In some embodiments, the composition comprises a total degradant concentration of less than about 2.5 wt. %. In some embodiments, the composition comprises a total degradant concentration of less than about 2 wt. %. In some embodiments, the composition comprises a total degradant concentration of less than about 1 wt. %.

The pharmaceutical composition can be used to treat and diseases, disorders and conditions that are responsive to treatment with agonists of the GC-C receptor. In some embodiments, methods are provided for treating gastrointestinal disorders in a patient (e.g., mammal or human) diagnosed with one or more gastrointestinal disorders or conditions, wherein the method comprises administering an effective amount of the composition or the oral dosage form to the patient. In some embodiments, methods are provided to use the compositions and oral dosage forms for treating gastrointestinal disorders including, but not limited to, GI motility disorders, irritable bowel syndrome, constipation-predominant irritable bowel syndrome (IBS-c), mixed-type irritable bowel syndrome (IBS-m), diarrhea predominant irritable bowel syndrome (IBS-d), chronic constipation, chronic idiopathic constipation, opioid induced constipation, post-surgical constipation (post-operative ileus), constipation associated with neuropathic disorders (e.g., constipation associated with Parkinson's Disease), constipation associated with cystic fibrosis or thyroid disease, dyspepsia (including functional dyspepsia or non-ulcer dyspepsia), gastroparesis, gastrointestinal motility disorders, functional gastrointestinal disorders, gastroesophageal reflux disease (GERD), inflammatory bowel disease, Crohn's disease, ulcerative colitis, functional heartburn, chronic intestinal pseudo-obstruction (or colonic pseudo-obstruction), visceral pain, abdominal pain, pelvic pain, pain associated with proctitis, fissures, anal fissure pain, vulvodynia, endometriosis, pain associated with endometriosis, prostatis, testicular pain, dysmenorrhea, pain associated with fibromyalgia, rectal pain from hemorrhoids, functional abdominal pain, interstitial cystitis pain, pain associated with venereal disease, diverticular diseases (including diverticulitis and pain associated with diverticulitis), and pain associated with celiac sprue. In some embodiments, methods are provided to use the compositions and oral dosage forms for treating disorders and conditions associated with constipation. In some embodiments, methods are provided to use the compositions and oral dosage forms for treating abdominal or visceral inflammation or pain associated therewith.

In some embodiments, a method is provided for treating chronic idiopathic constipation in a patient in need thereof by administering a solid oral dosage form described herein. In some embodiments, the solid oral dosage form comprises 72 μg of linaclotide. In some embodiments, the solid oral dosage form comprises 36 μg of linaclotide. In some embodiments, the solid oral dosage form comprises 18 μg of linaclotide. In some embodiments, the solid oral dosage form comprises 9 or 10 μg of linaclotide. In some embodiments, the solid oral dosage form is administered once daily in the morning at least 30 minutes before breakfast. In some embodiments, a method is provided for treating constipation predominant irritable bowel syndrome in a patient in need thereof by administering a solid oral dosage form described herein. In some embodiments, the solid oral dosage form comprises 72 μg of linaclotide. In some embodiments, the solid oral dosage form comprises 36 μg of linaclotide. In some embodiments, the solid oral dosage form is administered once daily in the morning at least 30 minutes before breakfast.

As used herein, a solid oral dosage form includes, without limitation, a tablet, a capsule, or a sachet or packet comprising the dry linaclotide composition. Tablets include, without limitation, those formulated to be swallowed whole, chewable tablets, orally disintegrating tablets, dissolvable tablets and effervescent tablets. Capsules include, without limitation, those formulated to be swallowed whole, or opened up and sprinkled or stirred into food or a beverage. Sachets include, without limitation, the solid form of the composition designed to be swallowed as a powder, sprinkled or stirred into food or a beverage, or dissolved in food or a beverage.

In some embodiments, a method is provided for increasing intestinal motility in a patient in need thereof, comprising administering an effective amount of the composition to the patient. Intestinal motility involves spontaneous coordinated dissentions and contractions of the stomach, intestines, colon and rectum to move food through the gastrointestinal tract during the digestive process.

In some embodiments, methods are provided for preventing a cancer or hyperplasia of the gastrointestinal tract or preventing reoccurrence of cancer or hyperplasia of the gastrointestinal tract in a patient in need thereof comprising administering an effective amount of the composition or the oral dosage form to the patient. In some embodiments, the cancer or hyperplasia is colorectal cancer, intestinal polyps or pre-cancerous growths or metastatic growths of gastrointestinal epithelial cells. In some embodiments, the composition or oral dosage form is administered simultaneously or sequentially with an effective amount of a COX-2 inhibitor. Examples of highly selective and selective COX-2 inhibitors include etoricoxib, rofecoxib, lumiracoxib, valdecoxib, celecoxib (Celebrex®), sulindac, diclofenac, meloxicam and etodolac. Non-selective NSAIDs that inhibit COX-2 include naproxen, ibuprofen, sodium salicylate and diflunisal. As used herein, the term “prevent” or “preventing” means to arrest, delay the onset (i.e., the period prior to clinical manifestation of a disease) or reoccurrence of cancer or hyperplasia, and/or reduce the risk of developing cancer or hyperplasia relative to a patient that has not been treated with a composition described herein.

In some embodiments, methods are provided for treating gastrointestinal disorders in pediatric patients with the compositions and oral dosage forms described herein. In some embodiments, methods are provided for treating gastrointestinal disorders in a pediatric patient diagnosed with one or more gastrointestinal disorders or conditions, wherein the method comprises administering an effective amount of the composition or the oral dosage form to the patient. In some embodiments, methods are provided to use the compositions and oral dosage forms for treating gastrointestinal disorders including, but not limited to, GI motility disorders, irritable bowel syndrome, constipation-predominant irritable bowel syndrome (IBS-c), mixed-type irritable bowel syndrome (IBS-m), diarrhea predominant irritable bowel syndrome (IBS-d), chronic constipation, chronic idiopathic constipation, opioid induced constipation, post-surgical constipation (post-operative ileus), constipation associated with neuropathic disorders, constipation associated with cystic fibrosis or thyroid disease, dyspepsia (including functional dyspepsia or non-ulcer dyspepsia), gastroparesis, gastrointestinal motility disorders, functional gastrointestinal disorders, gastroesophageal reflux disease (GERD), inflammatory bowel disease, Crohn's disease, ulcerative colitis , functional heartburn, chronic intestinal pseudo-obstruction (or colonic pseudo-obstruction), visceral pain, abdominal pain, pelvic pain, anal fissure pain, vulvodynia, endometriosis, and pain associated with endometriosis, prostatis, testicular pain, pain associated with fibromyalgia, rectal pain from hemorrhoids, functional abdominal pain, interstitial cystitis pain, diverticular diseases (including diverticulitis and pain associated with diverticulitis), and pain associated with celiac sprue. In some embodiments, methods are provided to treat IBS-c, IBS-m or chronic constipation (e.g., chronic idiopathic constipation) in pediatric patients with the compositions and oral dosage forms described herein. In some embodiments, methods are provided to treat IBS-c in a pediatric patient in need thereof. In some embodiments, methods are provided to treat chronic idiopathic constipation in a pediatric patient in need thereof.

In some embodiments, the oral dosage form is administered to a pediatric patient in need thereof as a tablet, capsule or sachet. In some embodiments, a sachet comprising the composition is opened and the contents are sprinkled on or stirred into food, such as applesauce, or into a beverage, such as water. In some embodiments, a capsule is swallowed whole with fluid, such as water, or is opened and sprinkled on or stirred into food or a beverage. Tablets may be swallowed whole, may be crushed and stirred into food or a beverage, or may be formulated as a chewable tablet.

In some embodiments, for example, the oral dosage form for a pediatric patient comprises from 1 μg to 90 μg of linaclotide. In some embodiments, for example, the solid oral dosage form comprises from 5 μg to 75 μg of linaclotide. In some embodiments, for example, the oral dosage form comprises 5 μg, 7.5 μg, 9 μg, 10 μg, 15 μg, 18 μg, 20 μg, 30 μg, 36 μg, 40 μg, 50 μg, 60 μg or 72 μg of linaclotide. In some embodiments, the oral dosage form comprises about 72 μg of linaclotide. In some embodiments, the oral dosage form comprises about 36 μg of linaclotide. In some embodiments, the oral dosage form comprises about 18 μg of linaclotide. In some embodiments, the oral dosage form comprises about 10 μg of linaclotide. In some embodiments, the oral dosage form comprises about 9 μg of linaclotide.

In some embodiments, the linaclotide composition may be formulated as a rectal dosage form for rectal administration. Rectal dosage forms include, without limitation, rectal suppositories, rectal foams or aerosols, enemas, rectal gels and rectal ointments. In some embodiments, the rectal dosage form may be administered to a patient in need thereof. In some embodiments, the rectal dosage form may be administered to a patient to treat abdominal or rectal pain, pain from anal fissures, ulcerative colitis, Crohn's disease or inflammatory bowel disease. In some embodiments, the rectal dosage form may be administered to a pediatric or geriatric patient. In some embodiments, the methods may comprise administering a therapeutically effective amount of the pharmaceutical composition to a patient in need thereof.

An effective amount of a composition comprising linaclotide or a pharmaceutically acceptable salt thereof required to achieve desired results (such as desired treatment and/or symptom relief) of a subject is dependent on several understood factors, such as the identity and severity of the disorder being treated, as well as the age, weight, etc., of the patient being treated.

A subject or patient in whom administration of the pharmaceutical composition is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions described herein are particularly suited for administration to any animal, particularly a mammal, and including, but by no means limited to, humans, rodents and non-rodents, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

In some embodiments, the unit dosage form and daily dose are equivalent. In some embodiments, the unit dosage form is administered with food at any time of the day, without food at any time of the day, with food after an overnight fast (e.g., with breakfast). In some embodiments, the unit dosage form is administered once a day, twice a day or three times a day. In some embodiments, one, two or three unit dosage forms will contain the daily oral dose of linaclotide. The precise amount of compound administered to a patient will be the responsibility of the attendant physician. However, the dose employed will depend on a number of factors, including the age and sex of the patient, the precise disorder being treated, and its severity. In some embodiments, the low-dose compositions can be used to produce higher unit dosage forms of linaclotide (e.g. 145 μg or 290 μg) in a single capsule or tablet.

In some embodiments, the compositions are administered as a monotherapy. In some embodiments, the composition consists essentially of an effective amount of linaclotide. In some embodiments, the composition consists of an effective amount of linaclotide.

In some embodiments, the compositions are directly administered to a patient, for example, in the form of a capsule, tablet or orally-disintegrating composition (e.g., orally-disintegrating tablet or film). In some embodiments, the compositions are dissolved, disintegrated and/or mixed on or within food or beverage prior to administration to patients (e.g., elderly or pediatric patients). In some embodiments, the composition is dissolved or disintegrated in a liquid, solution, or fluid optionally containing stabilizing agent(s), preservative(s), sweetener(s), or the like, etc. prior to administration to a patient (e.g., elderly or pediatric patient).

In other embodiments, the compositions are administered as part of a combination therapy. For example, a composition may be used in combination with other drugs or therapies that are used in the treatment, prevention, suppression, and/or amelioration of the diseases or conditions for which compounds of the invention are useful. The linaclotide can be co-administered or co-formulated with other medications. In one embodiment, the linaclotide composition can be co-administered with other medications used to treat gastrointestinal disorders including but not limited to acid suppressing agents such as Histamine-2 receptor agonists (H2As) and/or proton pump inhibitors (PPIs).

Such other drug(s) may be administered, by a route and in an amount commonly used therefore, contemporaneously or sequentially with a compound of the invention. When a compound of the present invention is used contemporaneously with one or more other drugs, a pharmaceutical unit dosage form containing such other drugs in addition to the compound of the invention may be employed. Accordingly, the pharmaceutical compositions of the present invention include those that also contain one or more other active components, in addition to a compound of invention.

Several methods can be used for evaluating the bioactivity of the linaclotide composition, including, but not limited to, immunoassays (e.g., enzyme-linked immunosorbent assay), radioimmuno assays, immunoradiometric assays, gel electrophoresis (e.g., SDS-PAGE), high performance liquid chromatography (HPLC), and/or high performance capillary electrophoresis (HPCE). In some embodiments, the bioactivity of the composition is assessed by a method comprising fixing linaclotide, incubating linaclotide with guanylate cyclase C (GCC), incubating GCC bound linaclotide with antibodies against GCC, incubating GCC antibody-bound linaclotide with fluorescently labeled antibodies against GCC antibodies, and detecting the linaclotide bound to the GCC antibodies by measuring the fluorescence intensity using a plate reader. The drug concentration can then be calculated based on the fluorescence reading of the solution.

For example, the bioactivity of the linaclotide compositions can be assessed and quantified using the following method, although other methods are available. The composition is added to a volumetric flask containing 60 ml of phosphate buffer having a pH of 4.5, and the flask is shaken for 60 minutes. 0.2 ml of the supernatant is then removed, and is added into one or more wells of a 96-well plate that is coated with GCC. The plate is sealed and incubated at 37° C. for 2 hr. At the end of incubation, the sample is removed and the plate is washed with phosphate buffered saline (PBS). The bound linaclotide is then incubated for 1 hour, at room temperature, with GCC (such as is available from Sigma-Aldrich Inc.) labeled with fluorescein isocyanate (FITC) in blocking buffer. After incubation, the well is washed with PBS. The fluorescence intensity of the end product is detected, for example, by using a plate reader. The linaclotide concentration is then calculated based on the fluorescence reading of the solution.

### Definitions

As used herein, unless otherwise indicated, “stabilizing agent” refers to a polymer, sterically hindered primary amine (e.g., amino acid), or cation (e.g., metal cation) component of the composition which is included in the composition in a stabilizing amount. For example, a polymeric stabilizing agent is a polymer that is included in the composition in a stabilizing amount. Similarly, a sterically hindered primary amine stabilizing agent is a sterically hindered primary amine that is included in the composition in a stabilizing amount. Moreover, a cationic stabilizing agent is a cation that is included in the composition in a stabilizing amount.

As used herein, unless otherwise indicated, “stabilizing amount” refers to a concentration, within the composition, of a polymer, sterically hindered primary amine (e.g., amino acid), or metal cation component at which the component increases the stability of linaclotide in the composition, as compared to a similar composition not having a stabilizing amount of the same component.

As used herein, unless otherwise indicated, a “low-dose pharmaceutical composition” is a pharmaceutical composition that comprises less than 100 μg of linaclotide, for example less than 90 μg, less than 80 μg, less than 75 μg, less than 70 μg, less than 60 μg, less than 50 μg, less than 40 μg, less than 30 μg or less than 20 μg of linaclotide.

As used herein, unless otherwise indicated, “therapeutically effective amount” means the amount of a linaclotide or a pharmaceutically acceptable salt thereof that, when administered to a mammal for treating a state, disorder or condition, is sufficient to effect a treatment (as defined below). The “therapeutically effective amount” will vary depending on the compound, the disease and its severity and the age, sex, weight, physical condition and responsiveness of the mammal to be treated. For example, a therapeutically effective amount of linaclotide, or its pharmaceutically acceptable salt or hydrate, can be an amount effective to treat gastrointestinal disorders, including irritable bowel syndrome, constipation-predominant irritable bowel syndrome, chronic constipation, opioid induced constipation and/or dyspepsia.

As used herein, unless other indicated, “pharmaceutically acceptable” means biologically or pharmacologically compatible for in vivo use in animals or humans, and preferably means, approved by a regulatory agency of the Federal or a state government or listed in the U.S. Pharmacopeia or other generally recognized pharmacopeia for use in animals, and more particularly in humans.

As used herein, unless otherwise indicated, the term “treat”, in all its verb forms, is used herein to mean to relieve, alleviate, and/or manage at least one symptom of a disorder in a subject.

The term “treatment” means the act of “treating” as defined above.

As used herein, unless otherwise indicated, the term “additives” refers to a pharmaceutically acceptable additive. Pharmaceutically acceptable additives include, without limitation, binders, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, unless otherwise indicated, an “excipient” is any pharmaceutically acceptable additive, filler, binder or agent.

As used herein, unless otherwise indication, “stressed conditions” refer to 40° C. and 75% relative humidity (RH).

As used here, unless otherwise indicated, the terms “about” and “approximately” mean within an acceptable error range for the particular value as determined by one of ordinary skill in the art, which will depend, in part, on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%. Alternatively, particularly with respect to biological systems or processes, the term can mean within an order of magnitude, preferably within 5-fold, and more preferably within 2-fold, of a value. Particular values are described in the application and claims, unless otherwise stated the term “about” means within an acceptable error range for the particular value.

All weight percentages (i e.,“% by weight” and “wt. %” and w/w) referenced herein, unless otherwise indicated, are measured relative to the total weight of the pharmaceutical composition.

The term “consisting essentially of”, and variants thereof, when used to refer to the composition, are used herein to mean that the composition includes linaclotide and other desired pharmaceutically inactive additives, excipients, and/or components (e.g., polymers, sterically hindered primary amines, cations, filling agents, binders, carriers, excipients, diluents, disintegrating additives, lubricants, solvents, dispersants, coating additives, absorption promoting additives, hydrolysis products, formaldehyde imine products, oxidation products, acetylation products, deamidation products, multimers, controlled release additives, anti-caking additives, anti-microbial additives, preservatives, sweetening additives, colorants, flavors, desiccants, plasticizers, dyes, or the like), and no other active pharmaceutical ingredient(s).

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

The following tests were employed in the examples section, unless otherwise indicated:

1) Stability of linaclotide compositions. For stability evaluation, linaclotide compositions (0.15 mg theoretical, actual 0.135 mg) were packaged into a HDPE bottle with desiccant, and stored under at 40° C. and 75% RH (“stressed conditions”). The amount of linaclotide was assayed initially and after up to 18 months of storage at stressed conditions. The concentration of linaclotide was analyzed and quantified using an HPLC method with the following mobile phase gradient: Mobile phase A: 50 mM of sodium perchlorate in a solvent containing 76% water and 24% acetonitrile and 0.1% of trifluoroacetic acid; Mobile phase B: 50 mM of sodium perchlorate in a solvent containing 5% water and 95% acetonitrile and 0.1% of trifluoroacetic acid; Flow rate: 0.6 ml/min; Column: YMC Pro C18, 150 mm×3 mm ID, 3 μm or equivalent; Column temperature: 40° C.; Fluorescence detection: excitation: 274 nm; emission: 303 nm; Injection volume: 100 μl.

2) Analysis of total degradants in the pharmaceutical composition: Degradant analysis was performed using an HPLC method employing the following conditions: Mobile phase A: Water: acetonitrile 98:2, with 0.1% (v/v) of trifluoroacetic acid; Mobile phase B: Water: acetonitrile 5:95, with 0.1% (v/v) of trifluoroacetic acid; Flow rate: 0.6 ml/min; Column: YMC Pro C18, 150 mm×3 mm ID, 3 μm or equivalent; Column temperature: 40° C.; UV detection: excitation: 220 nm; Injection volume: 50 μl. The percentage amounts of degradants in the composition were calculated by quantifying the area of all peaks in the HPLC chromatogram to obtain the “total peak area”, and dividing the peak area of each degradant by the total peak area. Specific degradants assayed include, for example, the hydrolysis product, Asp-7.

### Example 1

### Batch Formula Preparation of Linaclotide Beads

The manufacturing process consists of two stages: layering of the linaclotide drug substance, stabilizers and binder onto the beads and encapsulation of the linaclotide beads.

The linaclotide drug solution is produced by adding polyvinyl alcohol to heated purified water at 70-72° C. and mixing for 2 hours. After allowing the solution to cool, calcium chloride dehydrate is added to the solution under agitation and mixed for 10 minutes. L-histidine is added and mixed for 10 minutes. The solution is adjusted to pH 2.25 with hydrochloric acid, 36.5-38.0%. Sieved linaclotide is added and the solution is mixed for 60 minutes. Talc is then added and mixed for another 10 minutes.

The microcrystalline cellulose spheres are preheated in the fluid bed and then the linaclotide drug solution is sprayed onto the microcrystalline cellulose spheres at a target product temperature of 48° C. (45-52° C.). The product temperature is controlled by adjusting the inlet air temperature, spray rate, and process air volume, as needed in order to maintain the product temperature within the required range. The linaclotide beads are then dried in the fluid bed at the target product temperature of 48° C. (45-52° C.). The dried drug-layered beads are cooled, discharged and sieved.

The batch formula of linaclotide beads 145 μg/225 mg is provided in Table 1. The common linaclotide beads batch (25 kg) can be subdivided into smaller portions and used for the manufacture of the linaclotide capsules at various batch sizes and strengths based on the manufacturing requirements.

TABLE 1
Batch Formula for Linaclotide Beads, 145 μg/225 mg

| Component | Theoretical Quantity (Kg/Batch) |
|---|---|
| Linaclotide | 0.0161 |
| Calcium chloride dehydrate | 0.080 |
| Polyvinyl alcohol | 0.375 |
| L-histidine | 0.170 |
| Microcrystalline cellulose | 24.21 |
| spheres | |
| Talc | 0.150 |
| Purified water | 13.0 |
| Hydrochloric acid | 0.145 |
| (36.5-38.0%) | |
| Linaclotide beads, | 25.0 |
| 145 μg/225 mg | |

Purified water and hydrochloric acid are removed during processing.

### Example 2

### Description and Composition of the Linaclotide Capsules

Linaclotide capsules, 36 μg and 72 μg are compositionally proportional and are manufactured by filling the capsules with the common linaclotide beads 145 μg/225 mg. The batch formulas of linaclotide capsules, 36 μg and 72 μg are scale-independent and based on the encapsulation of linaclotide beads (capsule filling) per batch size up to 25 kg of linaclotide beads, 145 μg/225 mg. The theoretical batch formula of linaclotide capsules, 36 μg and 72 μg is provided in Table 2.

TABLE 2
Batch Formula for Linaclotide Capsules, 36 μg and 72 μg

| | Theoretical Quantity (Kg/Batch) | |
|---|---|---|
| | 36 μg Capsules | 72 μg Capsules |
| Component | (446,000 Capsules) | (223,000 Capsules) |
| Linaclotide beads, | 25.0 | 25.0 |
| 145 μg/225 mg | | |
| Empty gelatin capsule, | 27.2 | 13.6 |
| size 2 | | |
| Total Capsule Batch | 52.2 | 38.6 |
| Weight | | |

Linaclotide capsules, 36 μg and 72 μg are supplied in locked, size 2, white to off-white capsules with no imprint. The components and composition of linaclotide beads (145 μg/225 mg) and linaclotide capsules, 36 μg and 72 μg, are provided in Table 3 and Table 4. Linaclotide capsules are manufactured by filling size 2 gelatin capsules with the corresponding amounts of linaclotide beads to produce the finished dosage form. Actual weight is based on the assay of linaclotide drug substance.

TABLE 3
Components and Composition of Linaclotide Beads (145 μg/225 mg)

| | | Theoretical Weight (mg/capsule) | |
|---|---|---|---|
| Component | Function | 36 μg Capsules | 72 μg Capsules |
| Linaclotide | Drug substance | 0.036 | 0.072 |
| Calcium chloride | Stabilizer | 0.18 | 0.36 |
| dihydrate | | | |
| Polyvinyl alcohol | Stabilizer | 0.84 | 1.67 |
| L-histidine | Stabilizer | 0.38 | 0.76 |
| Microcrystalline | Bead core | 54.05 | 108.10 |
| cellulose spheres | | | |
| Talc | Processing aid | 0.33 | 0.67 |
| Linaclotide beads | Bead | 56 | 112 |
| (145 μg/225 mg) | | | |

| | | |
|---|---|---|
| Purified water | Processing | Removed during processing |
| | agent | |
| Hydrochloric acid | Processing | pH adjustment |
| (36.5-38.0%) | agent | |

TABLE 4
Components and Composition of Linaclotide Capsules, 36 μg and 72 μg

| | | Theoretical | | Theoretical | |
|---|---|---|---|---|---|
| | | Weight | | Weight | |
| | | (mg/capsule) | | (% w/w) | |
| Component | Function | 36 μg | 72 μg | 36 μg | 72 μg |
| Empty gelatin capsule | Capsule | 61.0^{a} | 61.0^{a} | 52.1 | 35.3 |
| size 2 | shell | | | | |
| Linaclotide beads | Beads | 56.0^{b} | 112.0^{b} | 47.9 | 64.7 |
| 145 μg/225 mg | | | | | |
| Total Capsule Weight | | 117.0 | 173.0 | 100.0 | 100.0 |

### Example 3

### Analytical Procedures and Results (Linaclotide Capsules, 36 μg and 72 μg)

The summaries of analytical test method and parameters used for the release and stability testing of linaclotide capsules are provided in this section.

### Assay, Content Uniformity and Identification A by UPLC Method

The identification, content uniformity and assay tests are determined against linaclotide reference standard using reverse-phase UPLC method with UV detection at 220 nm. The summary of method parameters is provided in Table 5.

TABLE 5
Summary of Test Method for Assay, Content Uniformity and
Identification

| Mobile phase A | 83:17:0.1 Water:Acetonitrile:Trifluoroacetic acid | | | |
|---|---|---|---|---|
| Mobile phase B | 95:5:0.1 Acetonitrile:Water:Trifluoroacetic acid | | | |
| Diluent | 0.1N Hydrochloric acid | | | |
| | Time | | | |
| Gradient profile | (minutes) | % A | % B | Comments |
| | 0-2 | 100 | 0 | Isocratic |
| | | | | hold |
| | 2-2.5 | 0 | 100 | Isocratic |
| | | | | cleaning |
| | | | | cycle |
| | 2.5-4.0 | 100 | 0 | Isocratic |
| | | | | equilibration |

| | |
|---|---|
| UV-detection | 220 nm |
| Injection volume | 10 μL |
| Run time | Approximately 3.5 minutes |
| Sample | 18-26 μg/mL |
| concentration | |
| Column | BEH C₁₈, 50 mm × 2.1 mm ID, |
| | 1.7 μm or equivalent |
| Column | 55° C. |
| temperature | |
| Autosampler | 4° C. |
| temperature | |
| Flow rate | 0.75 mL/min |

Stability Data

| | 72 μg | | 36 μg | |
|---|---|---|---|---|
| | 0 months | 72 μg | 0 months | 72 μg |
| Test | (Initial) | 3 months | (Initial) | 3 months |
| Total | 0.7 | 1.1 | 1.2 | 1.2 |
| Disulfide-Bonded | | | | |
| Multimers | | | | |
| Assay | 91.0 | 94.3 | 94.2 | 97.2 |
| Impurities | | | | |
| Asp⁷ and | 0.2 | 0.2 | 0.2 | 0.2 |
| Ala-insertion* | | | | |
| Trisulfide | None | <0.10 | None | <0.10 |
| | detected | | detected | |
| Des-Tyr¹⁴ | 0.1 | 0.2 | 0.1 | 0.2 |
| Cys¹-IMD | None | 0.3 | None | 0.3 |
| | detected | | detected | |
| Cys¹-Ketone | None | <0.10 | None | <0.10 |
| | detected | | detected | |
| Cys¹-N-Acetyl | 0.5 | 0.5 | 0.5 | 0.5 |
| Unspecified (each) | 0.15 | 0.33 | 0.15 | 0.31 |
| | (RRT | (RRT | (RRT | (RRT |
| | 0.80) | 0.773) | 0.80) | 0.773) |
| | 0.16 | | 0.15 | |
| | (RRT | | (RRT | |
| | 0.87) | | 1.24) | |
| Total (Specified | 1.1 | 1.6 | 1.1 | 1.5 |
| and Unspecified) | | | | |

*Ala-insertion refers to an impurity produced during manufacture of the peptide, which co-elutes with the Asp7 impurity. The Ala-insertion impurity is linaclotide with an additional alanine or an alanine isomer such as β-alanine inserted into the linear sequence of the peptide.

### Example 4

### Stability of Low-Dose Linaclotide Compositions

The low-dose linaclotide compositions were produced generally as described above in Examples 1 and 2. The low dose compositions were stored at 40° C./75% RH for six months and tested at 1, 2, 3, and 6 months for linaclotide content. Table 6 shows the batch formulations tested.

TABLE 6

| Bead Strength | Batch No | Batch Identity/Components |
|---|---|---|
| Linaclotide | BN00024691 | 1% PVA, histidine, talc 0.3% |
| Beads, | | Microcrystalline cellulose |
| 145 μg/225 mg | BN00024692 | 1.5% PVA, 0.6% talc, |
| | | microcrystalline cellulose |
| | BN00024695 | leucine, hydroxylpropyl methyl |
| | | cellulose, microcrystalline |
| | | cellulose |
| | BN00024694 | 1% PVA, 0% talc, |
| | | microcrystalline cellulose |

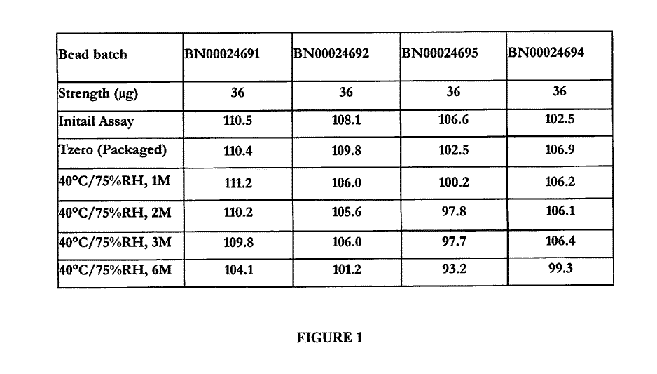

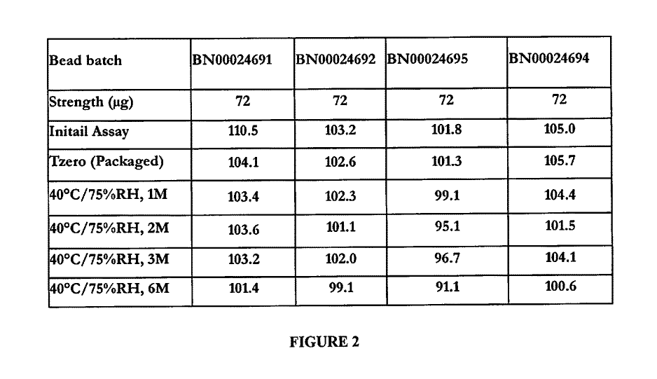

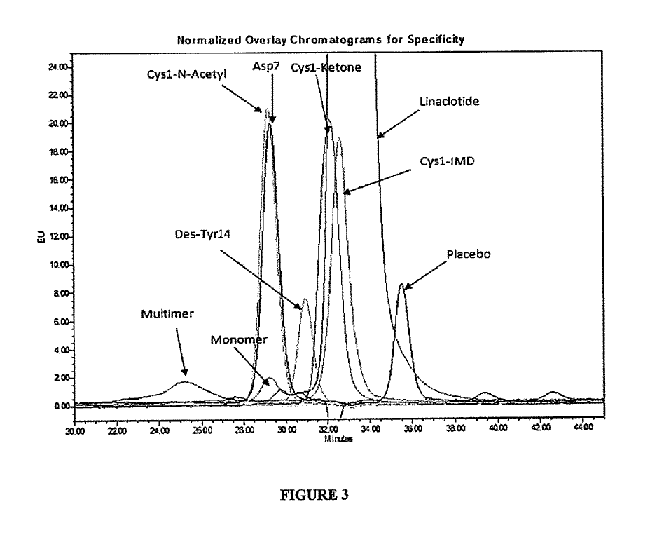

Linaclotide content and purity as well as the amount of linaclotide-related substances were measured essentially as described in Example 3. The results are provided in FIGS. 1 and 2. An example of an analysis of low-dose linaclotide compositions by HPLC is shown in FIG. 3, wherein the individual degradants are identified (e.g. Cys¹-IMD, Cys¹-N-Acetyl, Cys¹-Ketone, Asp⁷, Des-Tyr¹⁴, and multimers).

### Other Embodiments

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

What is claimed is:

1. A low dose pharmaceutical composition comprising linaclotide, Ca²⁺ and histidine.

2. The low dose pharmaceutical composition of claim 1, wherein the composition has a molar ratio of Ca²⁺:histidine of less than 2:1.

3. The composition of claim 1 or claim 2, wherein the composition further comprises a polymer.

4. The composition of claim 3, wherein the polymer is selected from polyvinyl pyrrolidone (PVP), polyvinyl alcohol (PVA) or a mixture thereof.

5. The composition of claim 1, wherein the composition comprises Ca²⁺ and histidine in a molar ratio of Ca²⁺:histidine between about 1:1 and 1:3.

6. The composition of claim 1, wherein the composition comprises Ca²⁺ and histidine in a molar ratio of Ca²⁺:histidine about 1:2.

7. A pharmaceutical composition comprising linaclotide; Ca²⁺; histidine; and polyvinyl alcohol (PVA), wherein the molar ratio of Ca²⁺:histidine:linaclotide is between 30-80:80-120:1.

8. The pharmaceutical composition of claim 7, wherein the Ca²⁺ is provided as CaCl₂.

9. A unit dosage form comprising the pharmaceutical composition of claim 8.

10. The unit dosage form of claim 7, wherein the linaclotide is present in the pharmaceutical composition in an amount between 1 μg to 100 μg.

11. The unit dosage form of claim 10, wherein the linaclotide is presented in an amount of 72 μg.

12. The unit dosage form of claim 10, wherein the linaclotide is presented in an amount of 36 μg.

13. The unit dosage form of claim 7, wherein the CaCl₂ is present in an amount of 180 or 360 μg.

14. The unit dosage form of claim 7, wherein the histidine is present in an amount of 380 or 760 μg.

15. The unit dosage form of claim 7, wherein the PVA is present in an amount of 840 or 1670 μg.

16. A pharmaceutical composition comprising coated beads, wherein the beads are coated with a coating solution comprising linaclotide, wherein the coating solution comprises:
linaclotide;
Ca²⁺;
histidine; and
polyvinyl alcohol (PVA),
wherein the molar ratio the of Ca²⁺:histidine:linaclotide is between 30-80:80-120:1.

17. A unit dosage form comprising the pharmaceutical composition of claim 16.

18. The unit dosage form of claim 17, wherein the linaclotide is present in the pharmaceutical composition in an amount between 1 μg to 100 μg.

19. The unit dosage form of claim 17, wherein the linaclotide is present in an amount of 72 μg.

20. The unit dosage form of claim 17, wherein the Ca²⁺ is provided as CaCl₂ in an amount of 180 or 360 μg.

21. The unit dosage form of claim 17, wherein the histidine is present in an amount of 380 or 760 μg.

22. The unit dosage form of claim 390, wherein the PVA is present in an amount of 840 or 1670 μg.

23. The pharmaceutical composition of claim 16, wherein the beads comprise microcrystalline cellulose.

24. A method of treating a gastrointestinal disorder comprising administering to a patient in need thereof, a therapeutically effective amount of the composition of claims 1-23.

25. The method of claim 24, wherein the gastrointestinal disorder is selected from the group consisting of irritable bowel syndrome, chronic constipation, opioid induced constipation and dyspepsia.

26. The method of claim 25, wherein the gastrointestinal disorder is chronic constipation.

27. The method of claim 25, wherein the gastrointestinal disorder is irritable bowel syndrome with constipation.

28. A method of making the composition of claims 1-23, comprising combining linaclotide with CaCl₂ and histidine, wherein the composition has a molar ratio of CaCl₂:histidine of less than 1:1.

29. A composition prepared by the method of claim 28.

30. A method of treating a gastrointestinal disorder comprising administering to a patient in need thereof, a therapeutically effective amount of the composition of claim 29.

31. The method of claim 30, wherein the gastrointestinal disorder is selected from the group consisting of irritable bowel syndrome, chronic constipation, opioid induced constipation and dyspepsia.

32. The method of claim 30, wherein the gastrointestinal disorder is chronic constipation.

33. The method of claim 30, wherein the gastrointestinal disorder is irritable bowel syndrome with constipation.
